(19) United States
(12) Patent Application Publication
(10) Pub. No.: US 2019/0282786 A1
(43) Pub. Date: Sep. 19, 2019

(54) CANNULA WITH PEELABLE OUTER LAYER

(71) Applicant: PACESETTER, INC., Sylmar, CA (US)

(72) Inventor: Aaron Raines, Dallas, TX (US)

(21) Appl. No.: 15/923,551

(22) Filed: Mar. 16, 2018

(51) Int. Cl. A61M 25/06; A61M 25/00

(52) U.S. Cl. CPC A61M 25/0668; A61M 2207/00; A61M 2025/0681; A61M 25/0009

(57) ABSTRACT

A cannula is provided that includes a core and a peelable outer layer. The core includes multiple longitudinal cannula body segments that collectively define a lumen and are generally maintained in position by the peelable outer layer. To remove the cannula from a patient, the outer peelable layer may be peeled and removed, thereby enabling the cannula segments to be separated and removed from the patient.

### TECHNICAL FIELD

Aspects of the present disclosure relate to cannulas for use in medical procedures and, in particular, cannulas for use in the delivery and insertion of implements into the body of a patient.

### BACKGROUND

A cannula is generally a tube that may be inserted into a patient for various medical purposes. For example, cannulas may be adapted to deliver medicine, to provide an insertion location for surgical instruments, or to drain fluids from within the body. Additionally, cannulas may be used to facilitate the implantation of a medical stimulation lead in a patient. In some instances, when inserting a device through a cannula wherein the proximal end of the device is larger than the distal end of device there is often a desire to remove the cannula from the device while leaving the device implanted in the patient. Therefore, ensuring a cannula can be inserted and removed from a patient with minimal difficulty while maintaining the target site for the implanted device is an important objective.

### SUMMARY

implementations described and claimed herein address the foregoing problems, among others, by providing a cannula including a rigid internal structure about which a peelable outer layer is disposed. In one implementation, the cannula includes an inner cannula body having a plurality of longitudinally extending cannula segments that collectively define a lumen. The cannula further includes a peelable outer layer encompassing a length of the inner cannula body.

In one example embodiment, the peelable outer layer may be heat shrunk onto the inner cannula body. The peelable outer layer may also include one or more longitudinal scores to facilitate peeling.

The outer peelable layer may include a first outer layer portion and a second outer layer portion abutting the first outer layer portion such that the first outer layer portion and the second outer layer portion form an interface along which the outer peelable layer may be split.

In certain embodiments, a first segment of the plurality of cannula segments may include a first engagement feature and a second segment of the plurality of segments may include a second engagement feature mated with the first engagement feature. For example, the first engagement feature may be a protrusion and the second engagement feature may be an indentation shaped or otherwise adapted to receive the protrusion.

In one preferred embodiment, cannula segments may be made from any rigid or semi-rigid material, such as, but not limited to metals and plastics depending upon user preference and the application thereof.

Additionally, the peelable outer layer may be formed from any peelable material, although good results have been observed by utilizing FEP shrinkable tubing for the peelable outer layer.

In another implementation of the present disclosure, a cannula is provided that includes a first longitudinal cannula segment and a second longitudinal cannula segment. The second longitudinal cannula segment abuts the first longitudinal such that the first and second longitudinal cannula segments define a lumen. The cannula further includes a peelable outer layer disposed about a length of the first and the second longitudinal cannula segments.

In one embodiment, the first longitudinal cannula segment may include one or more first engagement features. Each of the one or more engagement features shaped to engage a respective second engagement feature of the second longitudinal cannula segment, thereby preventing relative displacement between the first and second longitudinal cannula segments.

In certain embodiments, the peelable outer layer may be shrink wrapped onto the first and second longitudinal segments. The peelable outer layer may also include a split feature extending longitudinally along a length of the peelable outer layer and along which the peelable outer layer is predisposed to split. In one such embodiment, the peelable outer layer may include an outer layer body and a longitudinal strip such that the outer layer body and the longitudinal strip form an interface that acts as the spot feature.

In yet another implementation of the present disclosure a method of manufacturing a cannula is provided. The method includes disposed a plurality of longitudinally extending cannula segment to define a lumen and applying a peelable outer layer about a length of the plurality of cannula segments. Applying the peelable outer layer may include at least one of heat shrinking, overmolding, extruding, or co-molding the peelable outer layer onto the plurality of cannula segments.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWINGS

FIG. 1A is an isometric view of a first cannula according to the present disclosure including detail views of a proximal end and a distal end of the cannula.

FIG. 1B is an isometric view of the cannula of FIG. 1A in a peeled state including detail views of a proximal end and a distal end of the cannula.

FIGS. 2A and 2B are longitudinal side elevation view and a longitudinal cross-sectional view, respectively, of the cannula of FIG. 1A.

FIG. 3A is a lateral cross-sectional view of the cannula of FIG. 1A.

FIGS. 3B-3D are lateral cross-sectional views of a second, third, and fourth cannula, respectively, in accordance with the present disclosure.

FIG. 4 is a longitudinal side elevation view of a fifth cannula in accordance with the present disclosure.

FIG. 5 is a longitudinal side elevation view of a sixth cannula in accordance with the present disclosure.

FIG. 6A is a longitudinal side elevation view of an inner cannula body of the cannula of FIG. 1A in a separated and unpeeled state.

FIGS. 6B-6D longitudinal side elevation views of alternative inner cannula bodies, each of which are in a separated and unpeeled state.

FIG. 7 is a flow chart illustrating a method of manufacturing a cannula in accordance with the present disclosure.

### DETAILED DESCRIPTION

Aspects of the present disclosure involve cannulas and methods of manufacturing the same. Generally, a cannula in accordance with the present disclosure includes an inner cannula body that further includes multiple cannula segments. An outer peelable layer is applied to the cannula segments to maintain the cannula segments in alignment with each other. Following completion of a medical procedure, the outer peelable layer may be peeled from the cannula body and extracted from the patient. With the outer peelable layer removed, the cannula segments can be freely separated and similarly extracted from the patient.

Cannulas in accordance with the present disclosure include a cannula body about which an outer peelable layer is applied. The inner cannula body is formed from two or more cannula segments. These segments may generally be formed from a rigid or semi-rigid material, such as a metal or plastic, thereby providing structural strength to the cannula.

During use, the cannula is inserted into the body such that tabs or similar peeling features of the outer peelable layer remain accessible. When the cannula is to be removed after the completion of a medical procedure, the peeling features are pulled apart, thereby splitting the outer layer and enabling easy extraction of the outer layer. With the outer layer removed, the cannula segments may be separated from each other and individually extracted from the patient.

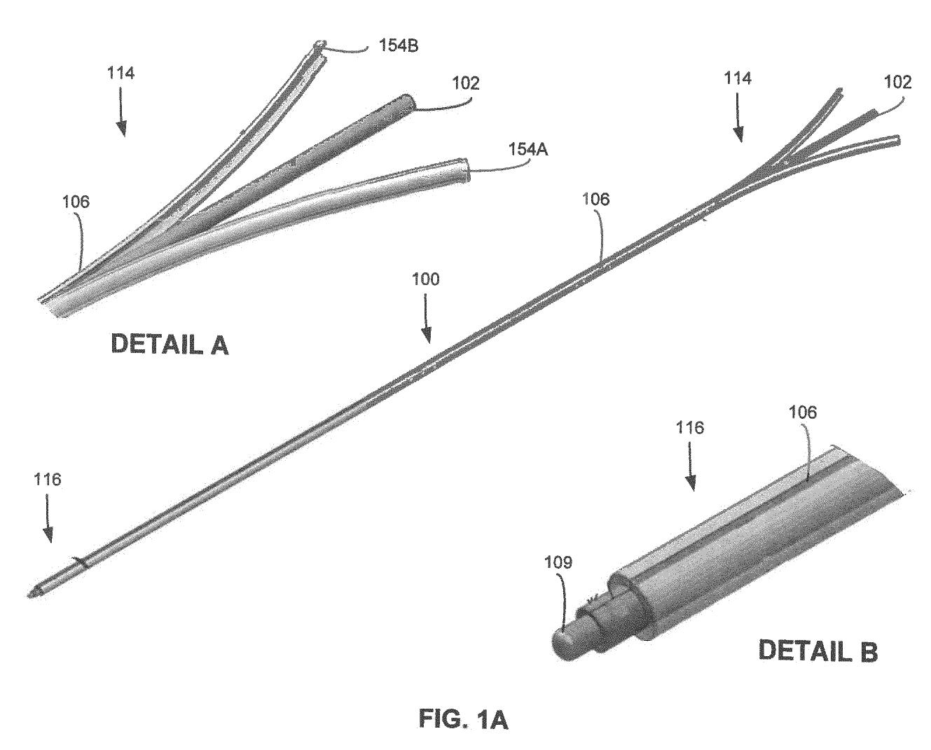

FIG. 1A is an isometric view of a cannula 100 in an unpeeled state according to one implementation of the present disclosure. The cannula 100 includes a proximal end 114 and a distal end 116, which are shown in further detail in Detail A and Detail B, respectively.

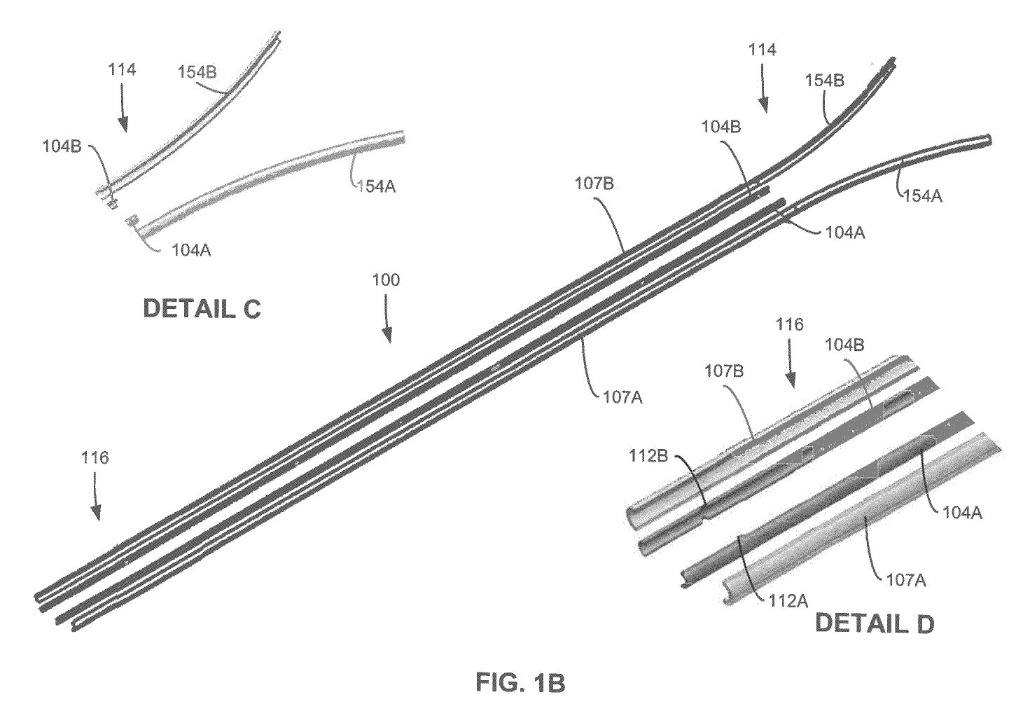

The cannula 100 includes an inner cannula body 102 about which an outer layer 106 is disposed. As shown in FIGS. 1A-1B, in certain implementations, the cannula body 102 includes two cannula segments 104A, 104B (shown in FIG. 1B) that, when aligned, define a lumen 110 (shown in FIGS. 2A-2B) extending through the cannula body 102. As illustrated in Detail B, the cannula body 102 may terminate in an atraumatic tip 109 to facilitate insertion of the cannula 100 into a patient. In certain implementations, for example, the atraumatic tip 109 may be an insert disposed at a distal end of the cannula body 102 during manufacturing that is removed after insertion into the patient but prior to insertion of any implements into the cannula 100. In other implementations, the atraumatic tip 109 may be split or ruptured by insertion of an implementation following insertion of the cannula 100 into a patient.

The cannula body 102 and, more specifically, the cannula segments 104A, 104B are preferably formed from a rigid or semi-rigid material such that the cannula segments 104A, 104B exhibit enough structural rigidity to facilitate the insertion of the cannula and other medical device into a patient. By way of example, it is preferred that the cannula segments 104A and 104B has such rigidity to facilitate the implantation of a deep brain stimulation lead into the brain of a patient.

Further details of the inner cannula body 102 and the first and second cannula segments 104A, 104B are now provided with reference to FIG. 1B, which is an isometric view of the cannula 100 in a peeled state, and Details C and D of FIG. 1B, which are detailed views of the distal end 116 and the proximal end 114 of the cannula 100, respectively. The first and second cannula segments 104A, 104B may include corresponding engagement features adapted to couple or otherwise maintain the cannula segments 104A, 104B in alignment with each other. For example, as illustrated in Detail D, the first cannula segment 104A may include a first engagement feature 112A adapted to mate with or otherwise couple with a corresponding second engagement feature 112B of the second cannula segment 104B. More specifically, the first engagement feature 112A is illustrated as a semi-circular tab extending from the first cannula segment 104A while the second engagement feature 112B is illustrated as a corresponding semi-circular indentation in second cannula segment 104B shaped to receive the semi-circular tab.

Cannulas in accordance with this disclosure may include one or more pairs of engagement features for coupling and maintaining alignment of cannula segments. For example, as shown in FIG. 1B, in addition to engagement features 112A, 112B, additional pairs of corresponding engagement features may be disposed along the length of the cannula segments 104A, 104B to provide further coupling between the cannula segments. Also, as illustrated in FIG. 2B, engagement features (such as engagement features 112C, 112D) may also be disposed on each edge of the cannula segments 104A, 104B.

Referring back to FIG. 1A, when the cannula 100 is fully assembled, the inner cannula body 102 is covered, at least in part, by an outer layer 106 such that the outer layer 106 maintains the cannula segments 104A, 104B in alignment. The outer layer 106 is adapted to be peelable meaning that the outer layer 106 may be split along its length into two or more strips. For example, in certain implementations, the outer layer 106 may include pull tabs or extensions, such as extensions 154A, 154B that may be pulled away from each other to cause the outer layer 106 to split. FIG. 1B illustrates the cannula 100 in a split state in which the outer layer 106 has been divided into two outer layer portions 107A, 107B. Once split, the outer layer 106 no longer maintains the cannula segments 104A, 104B in contact and the cannula segments 104A, 104B may be separated from each other, as illustrated in FIG. 1B.

The outer layer 106 is preferably formed from a relatively thin and pliable material that is sufficiently resilient to maintain the cannula segments 104A, 104B in contact but that may be split without significant effort or force applied by a physician or other medical personnel. In certain implementations, for example, the outer layer 106 is formed from FEP shrink tubing. The properties and performance of the outer layer 106 may also be altered by varying the thickness of the outer layer 106 with thicker outer layers providing increased resiliency and vice versa.

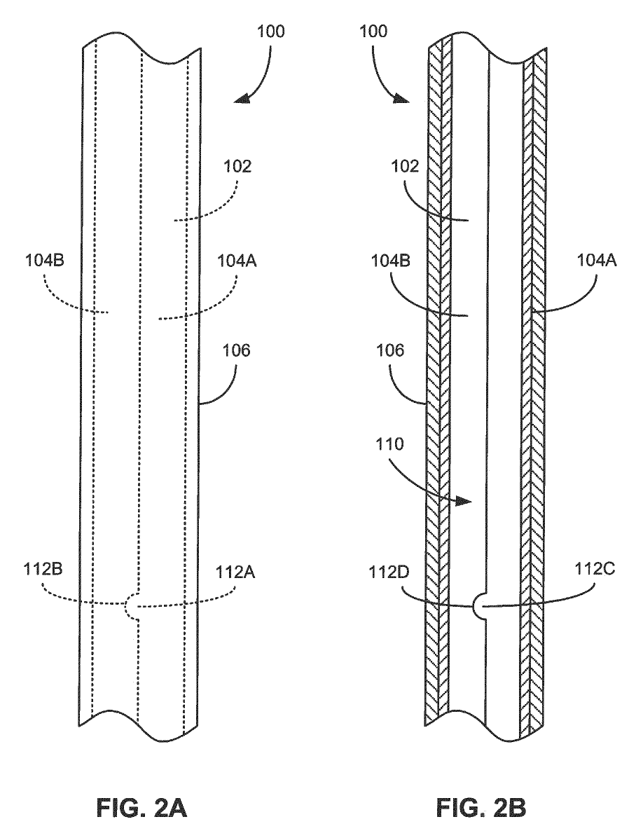

FIGS. 3A-3D are cross-sectional views of various cannulas in accordance with the present disclosure. FIGS. 3A-3D are generally intended to illustrate various arrangements of segments forming inner cannula bodies of the disclosed cannulas.

FIG. 3A is a cross-sectional view of the cannula 100 of FIGS. 1A-2B. The cannula 100 includes a first cannula segment 104A and a second cannula segment 104B that are enveloped by an outer layer 106. As shown in FIG. 3A, the first cannula segment 104A and the second cannula segment 104B may have substantially similar cross-sections. For example, each of the first cannula segment 104A and the second cannula segment 104B have semi-circular cross-sections such that when disposed against each other, the first cannula segment 104A and the second cannula segment 104B each define a respective half of a lumen 110.

FIG. 3B is a cross-sectional view of a second cannula 200 having an cannula body 202 including four cannula segments 204A-204D enveloped by an outer layer 206. As shown in FIG. 3B, each of the cannula segments 204A-204D have substantially similar cross-sections and, in particular, have quarter-round cross-sections. When made to abut each other, each of the cannula segments 204A-204D defines a respective quarter of a lumen 210.

Similarly, FIG. 3C is a cross-sectional view of a third cannula 300 having an inner cannula body 302 including three cannula segments 304A-304C enveloped by an outer layer 306 and having substantially similar cross-sections. When made to abut each other, each of the cannula segments 304A-304C defines a respective third of a lumen 310.

Although FIGS. 3A-3C illustrate cannulas having segments having substantially similar cross-sections, implementations of the present disclosure are not limited to such arrangements. For example, FIG. 3D is a cross-sectional view of a fourth cannula 400 having an inner cannula body 402 including three cannula segments 404A-404C enveloped by an outer layer 406. Unlike the cannula 300 of FIG. 3C in which each of the three cannula segments 304A-304C each formed a respective third of the inner cannula body 302, the cannula segments 404A-404C of the cannula 400 are illustrated in FIG. 3D as being uneven. Specifically, each of the cannula segments 404A and 404B form a respective quarter of the inner cannula body 402 while the cannula segment 404C forms half of the inner cannula body 402. Nevertheless, the cannula segments 404A-404C collectively define a lumen 410 extending through the inner cannula body 402.

The cannulas illustrated in FIGS. 3A-3D are intended only to illustrate examples of potential cannulas in accordance with the present disclosure and should not limit the present disclosure to any particular number or arrangement of cannula segments. Rather, cannulas in accordance with the present disclosure may include any suitable number of cannula segments, each of which may form any suitable portion of the inner cannula body.

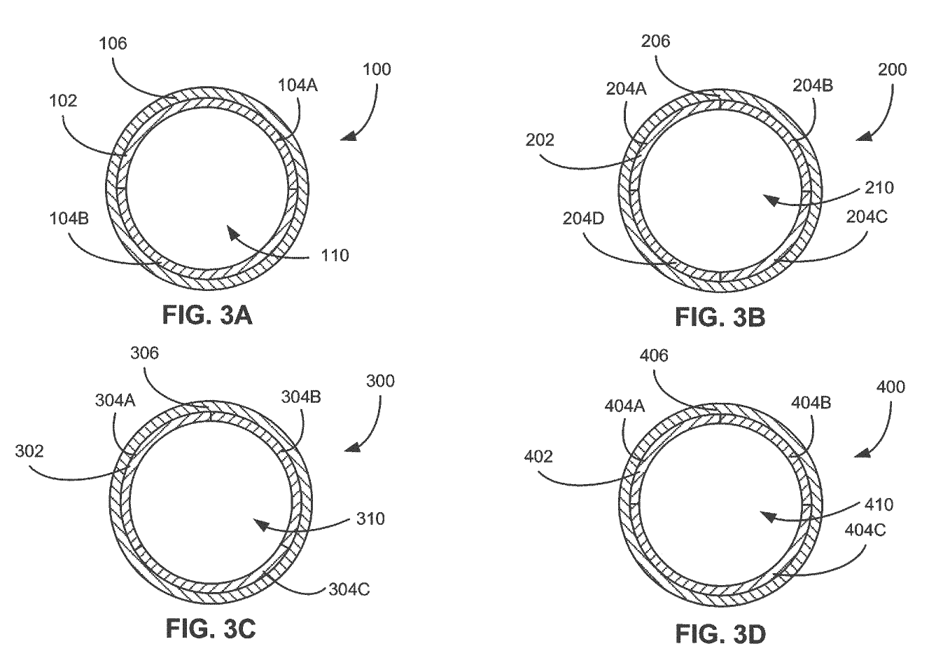

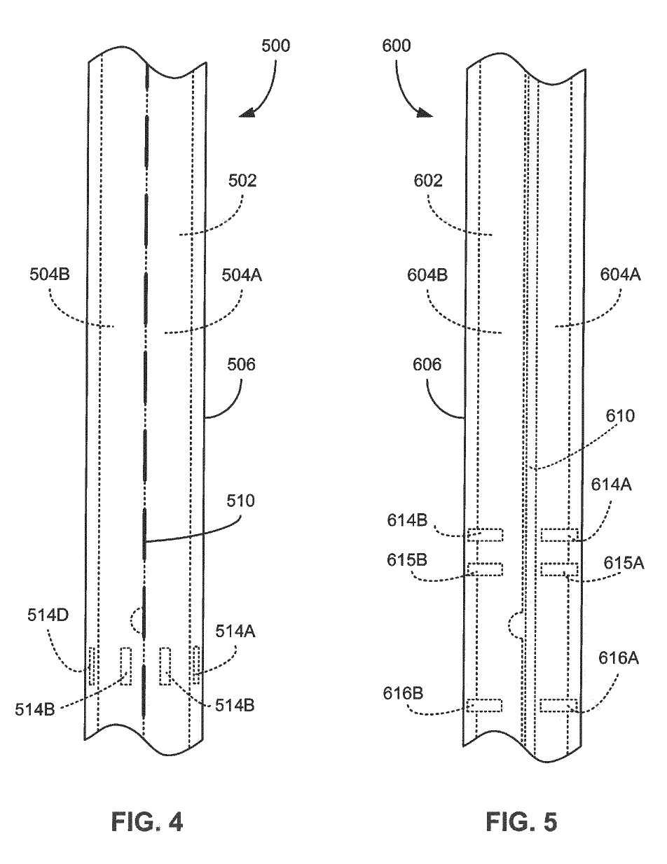

Cannulas in accordance with the present disclosure may also include various other features to improve their general utility and functionality. Examples of such features will now be discussed in the context of FIGS. 4 and 5 which are schematic illustrations of cannulas 500 and 600, respectively.

Referring first to FIG. 4, cannula 500 includes an inner cannula body 502 including each of a first cannula segment 504A and a second cannula segment 504B. At least a portion of the cannula segments 504A, 504B is enveloped by an outer layer 506 that may be peeled to allow separation of the cannula segments 504A, 504B.

In implementations of the present disclosure, the outer layer may include one or more features for facilitating peeling of the outer layer. Such features may make peeling of the outer layer easier and/or may direct peeling of the outer layer along a particular path or interface. For example, the outer layer 506 of FIG. 4 includes longitudinal scores 510 extending along the length of the outer layer 506. Such scores generally weaken the outer layer 506 such that when peeled, the outer layer 506 generally splits in line with the scores,

FIG. 5 illustrates another cannula 600 having an inner cannula body 602 including each of a first cannula segment 604A and a second cannula segment 604B. At least a portion of the cannula segments 604A, 604B is enveloped by an outer layer 606 that may be peeled to allow separation of the cannula segments 604A, 604B.

The cannula 600 also includes features for facilitating splitting of the outer layer 606. Specifically, the cannula 600 includes a longitudinal strip 610 disposed within or adjacent the outer layer 606. The longitudinal strip 610 may generally be formed of a rigid or semi-rigid material such that an interface is defined between the longitudinal strip 610 and the outer layer 606 along an outer edge of the longitudinal strip 610. Accordingly, when the outer layer 606 is peeled, the outer layer 606 generally splits along the interface between the longitudinal strip 610 and the outer layer 606.

Each of cannulas 500 and 600 of FIGS. 4 and 5 further include markers for locating the cannulas when inserted into a patient. For example, FIG. 4 includes a set of radiopaque markers 514A-514D molded into the outer layer 506 such that the radiopaque markers 514A-514D define a ring about the cannula 100. Similarly, the cannula 600 includes radiopaque half rings 614A-616A, 614B-616B disposed at various locations along the length of the outer layer 606. In either case, such radiopaque markers may be identified and monitored using a fluoroscope or similar device such that a physician may determine the location and/or orientation of the cannulas, thereby ensuring proper positioning prior to insertion of other implements into the patient.

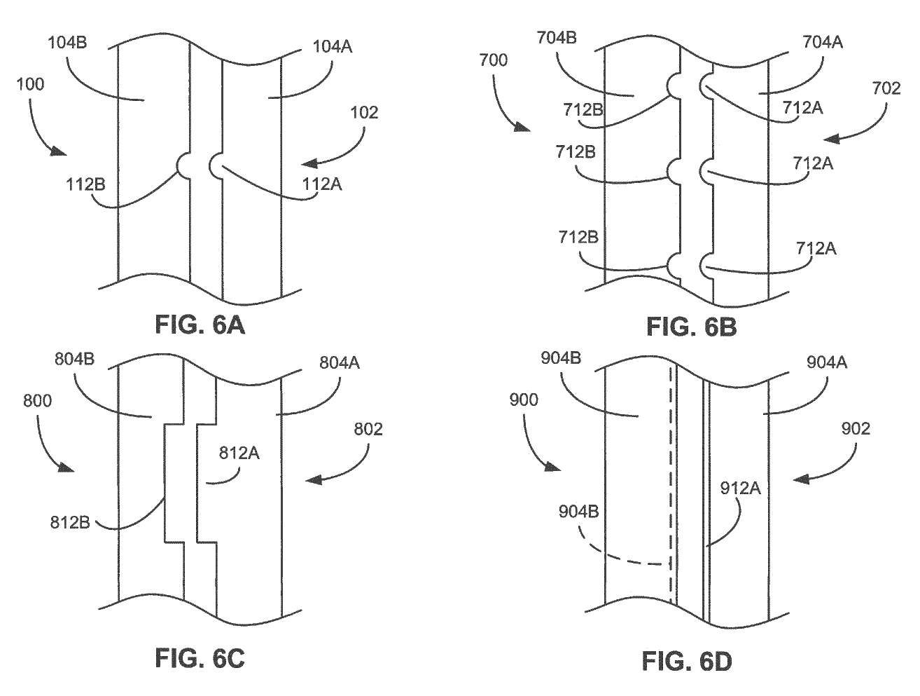

FIGS. 6A-6D illustrate various engagement features that may be used to fix the position of cannula segments, such as cannula segments 104A, 104B, relative to each other. In general, the engagement features are adapted to mate or otherwise engage such that the cannula segments are aligned relative to each other to facilitate the assembly of the two cannula segments 104A and 104B and at least partially restrict movement of the cannula segments relative to each other when assembled.

FIG. 6A is a partial view of the cannula 100 of FIGS. 1A-2B with the outer layer 106 removed and the cannula segments 104A, 104B partially separated. As shown, the first cannula segment 104A includes a first engagement feature 112A while the second cannula segment 104B includes a second engagement feature 112B. In particular, the first engagement feature 112A is illustrated as a semi-circular tab 112A while the second engagement feature 112B is illustrated as a corresponding semi-circular indentation 112B. When assembled, the semi-circular tab 112A is inserted into the semi-circular indentation 112B to align the first cannula segment 104A with the second cannula segment 104B and to prevent relative movement between the first cannula segment 104A and the second cannula segment 104B.

FIG. 6B illustrates an alternative cannula 700 including an inner cannula body 702 that further includes each of a first cannula segment 704A and a second cannula segment 704B. In contrast to the implementation illustrated in FIG. 6A, in which the cannula segment 104A, 104B were coupled by only a single pair of engagement features 112A, 112B, the implementation of FIG. 6B illustrates that cannulas in accordance with this disclosure may include multiple engagement features disposed along the lengths of the cannula segments 704A, 704B. For example, as shown in FIG. 6B, the first cannula segment 704A includes a plurality of first engagement features 712 in the form of semi-circular tabs 712A while the second cannula segment 704B includes a plurality of second engagement features 712B in the form of corresponding semi-circular indentations 712B shaped to receive respective ones of the first engagement features 712A. As shown in FIG. 6B, the first engagement features 712A and the second engagement features 712B are uniformly distributed along a length of the first cannula segment 704A and the second cannula segment 704B. However, in other implementations, engagement features may be distributed at any point along the length of the cannula segments. Similarly, while each of the first engagement features 712A and the second engagement features 712B shown in FIG. 6B are all the same (e.g., all semi-circular tabs and semi-circular indentations, respectively), engagement features for any given cannula segment may vary. For example, the cannula segments may include combination of indentations and tabs.

FIG. 6C is another alternative cannula 800 including an inner cannula body 802 that further includes each of a first cannula segment 804A and a second cannula segment 504B. The implementation of FIG. 6C illustrates a first alternative to the semi-circular tab/indentation engagement features of FIGS. 6A-6B. Specifically, the first cannula segment 804A includes a first engagement feature 812A in the form of substantially rectangular protrusion while the second cannula segment 704B includes a second engagement feature 712B in the form of a corresponding rectangular cutout.

In the previous examples of FIGS. 6A-6C, the engagement features of the cannula segments were generally illustrated as tabs and corresponding cutouts shaped to receive the tabs. In other implementations of the present disclosure, the engagement features of adjacent cannula segments may be adapted to couple or otherwise engage in other ways. For example, FIG. 6D is yet another alternative cannula 900 including an inner cannula body 902 that further includes each of a first cannula segment 904A and a second cannula segment 904B. In the implementation of FIG. 6D, the first cannula segment 904A includes a first engagement feature 912A in the form of a tongue extending at least partially along the length of the first cannula segment 904A and the second cannula segment 904B includes a second engagement feature 912B in the form of a groove extending along a corresponding length of the second cannula segment 904B. By inserting the tongue 912A of the first cannula segment 904A into the groove 912B of the second cannula segment 904B, the first and second cannula segments 904A, 904B may be coupled such that they are restricted from shifting longitudinally relative to each other.

The examples discussed in FIGS. 6A-6D generally include mating engagement features that are shaped to receive or otherwise couple to each other. Other examples of similar engagement features include, without limitation, mating lips, rabbets, or similar joints extending along corresponding segments of the cannula segments. In other implementations of the present disclosure, the cannula segments may be coupled together using a fastener or similar element separate from the cannula segments. For example, in certain implementations, the cannula segments may be maintained in position relative to each other by using one or more fastening elements such as, without limitation, one or more of bands, clips, rings, clamps, or similar fastening elements. The fastening elements may be adapted to extend around the cannula segments during assembly such that the cannula segments are maintained in position relative to each other, particularly during application of the peelable outer layer. In such implementations, the cannula segments may include grooves, protrusions, or similar structural features adapted to receive or otherwise align the fastening elements. After removal of the peelable outer layer, the cannula segments may be separated by first removing the fastening elements.

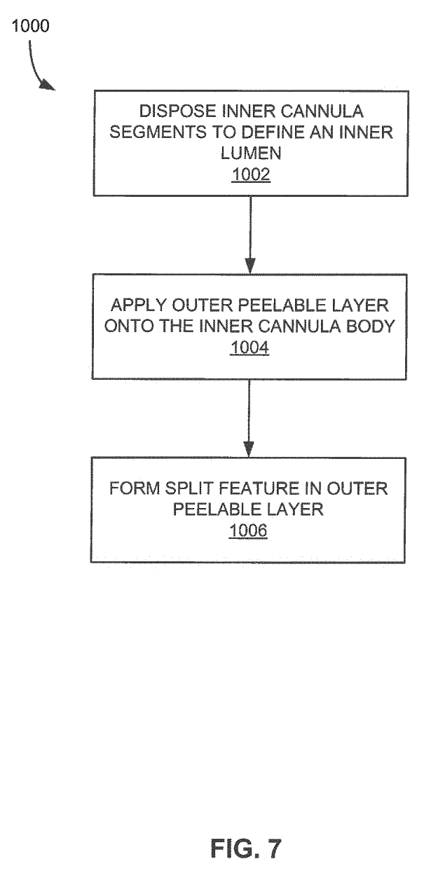

FIG. 7 is a flow chart 1000 illustrating an example method of manufacturing a cannula in accordance with the present disclosure. At operation 1002, the method includes disposing cannula segments to form an inner cannula body that defines a lumen. In certain implementations, disposing the cannula segments may include, among other things, mating, coupling, or otherwise engaging corresponding engagement features of the cannula segments, the engagement features adapted facilitate the alignment and assembly of the cannula segments and to maintain the cannula segments in alignment. Disposing the cannula segments may also include applying one or more fastening elements separate from the cannula segments, the fastening elements adapted to maintain the inner cannulas segments in alignment relative to each other.

Disposing the cannula segments may also include placing the cannula segments onto a jig, fixture, or similar support structure adapted to maintain the cannula segments in position during application of an outer peelable layer to the inner cannula body. In implementations in which the outer peelable layer is molded onto the inner cannula body, the support structure may be part of the mold used to apply the outer peelable layer to the inner cannula body.

At operation 1004, the outer pee/able layer is applied to the inner cannula body. The outer peelable layer may be applied in various ways including, without limitation, one of heat shrinking the outer peelable layer onto the inner cannula body, overmolding or co-molding the outer peelable layer onto the inner cannula body, and extruding the outer peelable layer about the inner cannula body. The process of applying the outer peelable layer to the inner cannula body may also include disposing radiopaque markers or similar elements within the outer peelable layer. For example, in certain implementations, radiopaque markers may be disposed within a mold adjacent the inner cannula body and subsequently molded in place during formation of the outer peelable layer.

At operation 1006, additional processing of the outer peelable layer may be applied, such as the formation one or more split features may be applied to the outer peelable layer. For example, such split features may be cut into the outer peelable layer using a blade, laser, or similar implement.

Various other modifications and additions can be made to the exemplary implementations discussed without departing from the spirit and scope of the presently disclosed technology. For example, while the embodiments described above refer to particular features, the scope of this disclosure also includes implementations having different combinations of features and implementations that do not include all of the described features. Accordingly, the scope of the presently disclosed technology is intended to embrace all such alternatives, modifications, and variations together with all equivalents thereof.

What is claimed is:

1. A cannula comprising:
an cannula body comprising a first and a second longitudinally extending segments, the first and second segments assembled to form a lumen between the first and second segments, the lumen extending the length of the cannula body;
the first segment including a first engagement feature and the second segment including a second engagement feature, with the first engagement feature and the second engagement feature being sized and shaped to mate together to facilitate the assembly of the first and second segments; and
a peelable outer layer disposed on an outer surface of the cannula body.

2. The cannula of claim 1, wherein the first engagement feature is a protrusion extending from an edge of the first segment and the second engagement feature is an indentation on the second segment, the indentation shaped to receive the protrusion of the first segment.

3. The cannula of claim 2, wherein the peelable outer layer comprises a first outer layer portion extending longitudinally along the cannula body and a second outer layer portion abutting the first outer layer portion to form an interface, the second outer layer portion extending longitudinally along the cannula body, such that the peelable outer layer is adapted to split along the interface.

4. The cannula of claim 3, wherein the peelable outer layer includes at least one longitudinal score between the first outer layer portion and the second outer layer portion.

5. The cannula of claim 4, wherein the peelable outer layer is heat shrunk onto the cannula body.

6. The cannula of claim 5, further comprising at least one radiopaque feature embedded in the peelable outer layer.

7. A cannula comprising;
a first longitudinal cannula segment;
a second longitudinal cannula segment abutting the first longitudinal cannula segment such that the first longitudinal cannula segment and the second longitudinal cannula segment define a lumen; and
a peelable outer layer disposed about a length of the first longitudinal cannula segment and the second longitudinal cannula segment.

8. The cannula of claim 7, wherein the first longitudinal cannula segment includes one or more first engagement feature and the second longitudinal cannula segment includes one or more second engagement features, each of the first engagement features engaged with a respective one of the second engagement features to prevent longitudinal displacement of the first longitudinal cannula segment relative to the second longitudinal cannula segment.

9. The cannula assembly of claim 8, wherein the peelable outer layer is shrink wrapped onto the first longitudinal cannula segment and the second longitudinal cannula segment.

10. The cannula assembly of claim 9, wherein the peelable outer layer includes a split feature extending longitudinally along a length of the peelable outer layer and along which the peelable outer layer is predisposed to split.

11. The cannula assembly of claim 10, wherein the peelable outer layer comprises an outer layer body and a longitudinal strip such that the outer layer body and the longitudinal strip define an interface, the split feature being the interface.

12. A method of manufacturing a cannula comprising:
disposing a plurality of longitudinally extending cannula segments to define a lumen; and
applying a peelable outer layer about a length of the plurality of cannula segments.

13. The method of claim 12, wherein applying the peelable outer layer about the plurality of cannula segments includes at least one of heat shrinking, overmolding, extruding, or co-molding the peelable outer layer onto the plurality of cannula segments.
